(19) United States
(12) Patent Application Publication
(10) Pub. No.: US 2018/0193289 A1
(43) Pub. Date: Jul. 12, 2018

(54) PROTOTYPICAL MOLECULAR PHARMACOLOGIC PROFILE OF FENDILINE AGAINST NEURODEGENERATIVE AND NEURODEVELOPMENTAL DISEASES

(71) Applicant: Alexandre Vamvakides, Ymittos Attikis (GR)

(72) Inventor: Alexandre Vamvakides, Ymittos Attikis (GR)

(21) Appl. No.: 15/381,338

(22) Filed: Dec. 16, 2016

(51) Int. Cl. A61K 31/137; A61P 25/28; A61P 25/16

(52) U.S. Cl. CPC A61K 31/137; A61P 25/16; A61P 25/28

(57) ABSTRACT

The present invention concerns the prototypical pharmacologic profile of 3,3-diphenyl-N-(1-phenylethyl) propan-1-amine (Fendiline), its enantiomers and its pharmaceutically acceptable salts characterized by their selective sigma-1 receptor affinity (vs, sigma-2 receptor: 14 to 18 nM vs 730 to 850 nM) and, therefore, prototypical allosteric antagonisms of brain N-methyl-D-aspartate receptors [NMDA(−)], sodium channels [Nav(−)] and discreet desensitization of muscarinic receptors [M(−)]. Of interest is the positive allosteric modulation of the γ-aminobutyric acid A (GABA) (A) and GABA (B) receptors, originating brain pro-GABAergic and anti-glutamatergic activities and also an unique pharmacological profile which antagonize all the components of the vicious circle generated in the brain by Glutamate/GABA imbalance, with protective and preventive action against all the behavioral abnormalities, the cognitive deficits and neurotoxicities, in neurodegenerative (Alzheimer's, Parkinson's Huntington's and Multiple Sclerosis) diseases and neurodevelopmental diseases (Autism spectrum disorders and related syndromes).

### BACKGROUND OF THE INVENTION

### Field of the Invention

The present invention concerns the prototypical pharmacological profile of 3,3-diphenyl-N-(1-phenylethyl) propan-1-amine (Fendiline) and the new putative brain preventive properties, against neurodegenerative and neurodevelopmental diseases. The present invention relates to a prototypical profile of Fendiline for use in connection with at least preventive activity against the neurodegenerative and neurodevelopmental diseases.

### SUMMARY OF THE INVENTION

The present invention provides a novel prototypical pharmacological profile of 3,3-diphenyl-N-(1-phenylethyl) propan-1-amine (Fendiline) and the new putative brain preventive properties, against neurodegenerative and neurodevelopmental diseases, and overcomes the above-mentioned disadvantages and drawbacks of the prior art. As such, the general purpose of the present invention, which will be described subsequently in greater detail, is to provide a new and novel prototypical pharmacological profile of 3,3-diphenyl-N-(1-phenylethyl) propan-1-amine (Fendiline) and the new putative brain preventive properties, against neurodegenerative and neurodevelopmental diseases and method which has all the advantages of the prior art mentioned heretofore and many novel features that result in a prototypical pharmacological profile of 3,3-diphenyl-N-(1-phenylethyl) propan-1-amine (Fendiline) which is not anticipated, rendered obvious, suggested, or even implied by the prior art, either alone or in any combination thereof.

To attain this, the present invention essentially comprises a compound comprising an effective amount of 3,3-diphenyl-N-(1-phenylethyl) propan-1-amine (Fendiline), its enantiomers and pharmaceutically acceptable salts or combinations thereof. The compound can be prepared with brain protective and preventive activities and used against neurodegenerative and neurodevelopmental diseases. The compound can be selective sigma-1 receptor ligands and, therefore, prototypical allosteric antagonists of N-methyl-D-aspartate receptors [NMDA(−)], sodium channels [Nav(−)] and discreet desensitization agents of muscarinic receptors [M(−)], exercing also positive allosteric modulations on brain γ-aminobutyric acid (B) [GABA (B) (+)] and [GABA (A) (+)] receptors.

It can be appreciated that the present invention essentially comprises a pharmaceutical composition comprising an effective amount of a compound and at least one pharmaceutically acceptable excipient. The pharmaceutical composition can be prepared with brain protective and preventive activities and used against neurodegenerative and neurodevelopmental diseases. The compound can be selected from the group consisting of 3,3-diphenyl-N-(1-phenylethyl) propan-1-amine (Fendiline), enantiomers and pharmaceutically acceptable salts of Fendiline, and combination thereof. The compound can be as prototypical sigma-1 ligand with [NMDA(−)], [Nav(−)], [M(−)], [GABA(B)(+)] and [GABA(A)(+)].

It can be appreciated that the present invention essentially comprises a method of using a pharmaceutical composition including preparing a pharmaceutical composition comprising an effective amount of a compound and at least one pharmaceutically acceptable excipient. The compound can be selected from the group consisting of 3,3-diphenyl-N-(1-phenylethyl) propan-1-amine (Fendiline), enantiomers and pharmaceutically acceptable salts of Fendiline, and combination thereof. The compound can be as prototypical sigma-1 ligand with [NMDA(−)], [Nav(−)], [M(−)], [GABA(B)(+)] and [GABA(A)(+)]. An effective amount of the pharmaceutical composition can be administered to an individual suffering from neurodegenerative or neurodevelopmental disease. The neurodegenerative or neurodevelopmental disease can be selected from the group consisting of Alzheimer's disease, Huntington's disease, Parkinson's disease, Multiple Sclerosis, Autism spectrum disorders and brain neurodegenerative or neurodevelopmental syndromes originated therefrom, respectively, late or early stage human life, and brain Glutamatergic/GABA imbalance.

There has thus been outlined, rather broadly, features of the invention in order that the detailed description thereof that follows may be better understood and in order that the present contribution to the art may be better appreciated.

The invention may also include the effective amount administered to an individual suffering from the neurodegenerative or neurodevelopmental disease can be 50 to 300 mgs (po), daily. There are, of course, additional features of the invention that will be described hereinafter and which will form the subject matter of the claims attached.

Numerous objects, features and advantages of the present invention will be readily apparent to those of ordinary skill in the art upon a reading of the following detailed description of the present invention, but nonetheless illustrative, embodiments of the present invention when taken in conjunction with the accompanying drawings.

As such, those skilled in the art will appreciate that the conception, upon which this disclosure is based, may readily be utilized as a basis for the designing of other structures, methods and systems for carrying out the several purposes of the present invention. It is, therefore, that the claims be regarded as including such equivalent constructions insofar as they do not depart from the spirit and scope of the present invention.

It is therefore an object of the present invention to provide a new and novel prototypical pharmacological profile of Fendiline that has all of the advantages of the prior art and none of the disadvantages.

It is another object of the present invention to provide a new and novel prototypical pharmacological profile of Fendiline that may be easily and efficiently manufactured and marketed.

An even further object of the present invention is to provide a new and novel compound or pharmaceutical composition of Fendiline that has a low cost of manufacture with regard to both materials and labor, and which accordingly is then susceptible of low prices of sale to the consuming public, thereby making such compound or pharmaceutical composition of Fendiline economically available to the buying public.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated embodiments of the invention.

### DETAILED DESCRIPTION OF THE INVENTION

The present invention concerns the prototypical profile and the putative preventive properties of Fendiline against the neurodegenerative or neurodevelopmental diseases. Fendiline as referred to in the present invention, may include compounds having the general formula A: 3,3-diphenyl-N-(1-phenylethyl) propan-1-amine (Fendiline).

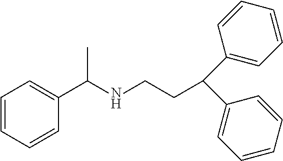

The Fendiline's profile was characterized with new putative brain preventive properties, against neurodegenerative and neurodevelopmental diseases, of this compound characterized by a highly selective sigma-1(σ1) receptor affinity (vs sigma-2: 14 to 18 nM vs 730 to 850 nM) and low intrinsic activity, exercing, therefore, partial agonistic activity at low doses (0,003 to 1.0 mg/kg per os (po), in mice) and antagonistic, at more than 1.0 mg/kg (po) in mice. In good agreement with its antagonistic σ1 properties, Fendiline exhibited allosteric positive modulation on the γ-aminobutyric acid (GABA) A and GABA (B) receptors as well as, σ1 induced, negative allosteric modulations of the N-methyl-D-aspartic acid (NMDA) glutamatergic receptors, the sodium channels and discreet negative modulation of the cholinergic muscarinic receptors.

Of particular interest is the above, σ1 induced, positive allosteric modulation of the GABA (B) receptors, exercing a corrective presynaptic inhibition of the brain glutamatergic pathways and GABAergic interneurons, which exhibited high aberrant activation in neurodegenerative diseases, principally in Alzheimer's disease (AD) but also in Huntington's chorea (HC), Parkinson's disease (PD), or Multiple Sclerosis (MS) as well as in neurodevelopmental diseases like Autism Spectrum Disorders (ASDs), in which the aberrant glutamatergic activation induced seizures (usually without convulsions) and GABAergic interictal activity (mostly via GABAergic interneurons) originating cognitive deficits and various pathological profiles (AD, HC, PD, MS, ASDs), according to the brain regions (Hippocampus, Basal Ganglia, Cortex) affected by this aberrant activations, characterized by seizures and progressive neurodegenerations.

Moreover, Fendiline, by its negative modulation of the brain glutamatergic pathways and its discreet, σ-1 induced, negative modulation of the muscarinic receptors, exerced an efficient control against the brain cholinergic aberrant hyperactivation and the resulting altered theta brain oscillations and cognitive deficits.

Fendiline exhibited a molecular pharmacologic profile with putative preventive properties against the pathophysiological components of the above diseases, characterized by similar brain neurodegenerative phenomena appearing, respectively, in the late or early stages of the human life. Despite a variety in the factors triggering these diseases (genetic, pathological or environmental) their evolution follows similar processes, i.e., —(1) reinforced glutamatergic activity in the brain and disturbed Glutamate/GABA balance inducing aberrant hyperactivation of the GABAergic interneurons in the striatum, nucleus accumbens and, particularly, in the cortex, with downregulation of the big GABAergic pathways: striato-nigral and pallido-ventral tegmental area (VTA).

Downregulation of the above big GABAergic pathways induce the activation of the ascending dopaminergic pathways to striatum, nucleus accumbens and cortex releasing dopamine (DA), the oxidative metabolism of which will reduce the redox site of the extrasynaptic glutamatergic NMDA receptors, then activating them and amplifying the Glutamate/GABA imbalance of the brain.

A forward analysis of the resulting new dynamics (in the brain glutamate, GABA, cholinergic and DA pathways) triggered by the Glutamate/GABA imbalance and also the aberrant hyperactivation of the GABA interneurons, make it easy to conclude that, —(2) the initiating factors (soluble A(3, anoxia, autoimmune reactions, toxics) trigger a vicious circle, which will be continuated with the NMDA induced aberrant activation of the cholinergic interneurons releasing acetylcholine (Ach), which, by its action on the astroglial nicotinic receptors, will release glutamate, amplify the Glutamate/GABA imbalance and initiate neurodegenerative processes. Moreover, —(3) the vicious circle will be aggravated by the extrasynaptic glutamatergic and cholinergic stimulations of the GABAergic interneurons in the striatum, nucleus accumbens and cortex, which will amplify the downregulation of the striato-nigral and accumbal-VTA GABAergic pathways with further disinhibition of the ascending DA pathways (nigrostriatal, VTA-nucleus accumbens and VTA-cortex) potentiating the NMDA receptors and the aberrant glutamatergic activity in striatum, nucleus accumbens and cortex, on the one hand, and, on the other, amplifying the inhibitory effects of the nigral GABAergic projections to thalamus and reticular formation which will allow the propagation of the aberrant synchronous cortical glutamatergic hyperactivity and the frequently observed seizures, usually without convulsions but accompanied by cognitive deficits, in the neurodegenerative and neurodevelopmental diseases.

It is, therefore, evident that all the described negative behavioral and cognitive consequences, obtained in the appropriate in vivo animal models of the above diseases, derived from the described vicious circle, i.e., following genetic or toxic environmental factors, autoimmune reactions and/or hypoxia, the glutamatergic (NMDA) activation, in the neonatal stage of life could initiate the above vicious step (1) and anxiety, fear, social disconnection, compulsive reactions, agressivity and, DA induced, hypermotility and stereotypies, when amplified in step (2), and seizures, lethargy and cognitive deficits, with step (3), in neurodevelopmental diseases and ASD.

In neurodegenerative diseases and, in particular AD, soluble Aβ, GRKS deficits and/or hypoxia induce step (1). However, step (2) of the vicious circle is usually expressed by, DA induced, delirant or psychotic symptoms. In PD and HC, the amplified aberrant glutamatergic activity in steps (2/3) caused, respectively, tremor or dyskinesias (motor and orofacial) induced by the neurotoxic aberrant glutamatergic hyperactivity on, respectively, striatal DA or GABA neurons, while in MS it caused moving disturbances, anxiety, unstable mood, depression, seizures cognitive deficits and neuropathic pain.

Concerning the neuropathic pain, there is presently evidence that it is a frequent manifestation of all the neurodegenerative diseases.

Fendiline, via its molecular pharmacological properties could antagonise all the components and steps of the above described vicious circle. Firstly, by its sigma-1 induced negative modulation of the NMDA receptors, Fendiline could inhibit the activation of the GABAergic and cholinergic interneurons and disinhibit the striatonigral and pallido-VTA big GABAergic pathways, annulling the aberrant activation of the ascending dopaminergic pathways, inhibiting the excessive DA release and the NMDA receptors potentiation. Moreover, Fendiline induced positive allosteric modulation of the GABA (B) receptors, situated presynaptically on the glutamatergic neurons, which could train an inhibitory action against the aberrant glutamatergic activation. In addition, Fendiline, by its positive allosteric modulation of GABA (A) and GABA (B) receptors develop an efficient nigral inhibition of the efferent GABAergic pathways projecting to thalamus and reticular formation, disinhibiting and disynchronising the latter, arresting the propagation of the hypersynchronous glutamatergic cortical hyperactivity and the induced seizures.

Finally, Fendiline, by inhibition of the aberrant activation of GABAergic and cholinergic interneurons could prevent the cognitive deficits and neurodegenerations.

In good agreement with the above receptors affinities and its molecular pharmacological properties, Fendiline, at 1.0 to 50 mg/kg, (po), in mice antagonized all the behavioral effects (in mice) corresponding to the symptoms, in humans, announcing the development of the neurodegenerative and neurodevelopmental diseases, i.e., anxiety, fear, social disconnection, agressivity, compulsive reactions, hypermotility, stereotypic activity, pain, lethargy and seizures, appearing at various degrees according to the amplitude of the Glutamate/GABA imbalance and to the brain regions (Basal Ganglia, Hippocampus, Cortex) affected by the above imbalance. Indeed, all the above behavioral effects (obtained, in mice, by administration of the GABAergic antagonist Pentylenetetrazole (PTZ) at 10 to 70 mg/kg, intraperitoneal (ip): with 10 to 40 mg/kg (ip) giving fear, anxiety, social disconnection, and agressivity, hypermotility and stereotypies—the latter being better expressed by previous (reserpine induced) hypersensibilisation of the brain DA receptors—and seizures (obtained at 50 to 70 mg/kg (ip) of PTZ), were antagonized by Fendiline at 1.0 to 50 mg/kg (po).

It is noteworthy, that Fendiline did not protect against the PTZ clonic convulsions and the fatal tonic crises (tonic extensions), in Maximal Electrochock (IVIES) or high doses (more than 80 mg/kg (ip), in mice) of PTZ, clearly indicating its highly interesting GABA (B) presynaptic inhibitory activities, on brain glutamatergic pathways and GABAergic interneurons, by controlling, respectively, their aberrant hyperactivation and the generation of interictal spikes and cognitive deficits.

Moreover, the Fendiline induced GABA (B) presynaptic inhibitory control on the aberrant hyperactivation of the glutamatergic pathways constitute an efficient negative control of the cholinergic interneurons which, in association with the Fendiline's discreet, sigma-1 induced, negative control of the muscarinic receptors, allow an efficient protection against the altered theta brain oscillations and cognitive deficits, resulting from the above described vicious circle.

It is, therefore, evident that Fendiline could not be envisaged as putative antiepileptic drug, because, the above described GABA (B) presynaptic inhibitory controls, limited also its hyperactivation of the big GABAergic nigrostriatal neurons, which play a determinant role for the obtention of an efficient antiepileptic activity.

However, Fendiline increased significantly the latency of PTZ clonic convulsions by 50% to 67% and exhibited an important increase (2 to 10 fold) of the lethality latency, after the PTZ fatal tonic crises, with also 10% to 30% of total (survival) protection, when Fendiline was administered at 20 to 50 mg/kg (po) in mice, clearly indicating its protective effect against the, brain seizures induced, mitochondrial toxicity, which, chronically, lead to neurotoxicity and neuronal apoptosis, in the neurodevelopmental and neurodegenerative diseases.

In relation with the above, seizures induced, neurotoxic processes, neuropathic pain is a frequent manifestation in all the neurodegenerative diseases and the author has recently demonstrated, in the appropriate animal models, the analgesic effect of Fendiline against this component of the (early or late stage life) neurodegenerative diseases.

It can be appreciated that a compound comprising an effective amount of 3,3-diphenyl-N-(1-phenylethyl) propan-1-amine (Fendiline), its enantiomers and pharmaceutically acceptable salts or combinations thereof, can be prepared with brain protective and preventive activities and used against neurodegenerative and neurodevelopmental diseases. The compound can be selective sigma-1 receptor ligands and, therefore, prototypical allosteric antagonists of N-methyl-D-aspartate receptors [NMDA(−)], sodium channels [Nav(−)] and discreet desensitization agents of muscarinic receptors [M(−)], exercing also positive allosteric modulations on brain γ-aminobutyric acid (B) [GABA (B) (+)] and [GABA (A) (+)] receptors.

It can be appreciated that a pharmaceutical composition comprising an effective amount of a compound and at least one pharmaceutically acceptable excipient, can be prepared with brain protective and preventive activities and used against neurodegenerative and neurodevelopmental diseases. The compound can be selected from the group consisting of 3,3-diphenyl-N-(1-phenylethyl) propan-1-amine (Fendiline), enantiomers and pharmaceutically acceptable salts of Fendiline, and combination thereof. The compound can be as prototypical sigma-1 ligand with [NMDA(−)], [Nav(−)], [M(−)], [GABA(B)(+)] and [GABA(A)(+)]. The effective amount can be 50 to 300 mgs (po), administered daily to an individual suffering from neurodegenerative or neurodevelopmental disease.

It can be appreciated that a method of using a pharmaceutical composition can include preparing a pharmaceutical composition comprising an effective amount of a compound and at least one pharmaceutically acceptable excipient. The compound can be selected from the group consisting of 3,3-diphenyl-N-(1-phenylethyl) propan-1-amine (Fendiline), enantiomers and pharmaceutically acceptable salts of Fendiline, and combination thereof. The compound can be as prototypical sigma-1 ligand with [NMDA(−)], [Nav(−)], [M(−)], [GABA(B)(+)] and [GABA(A)(+)]. An effective amount of the pharmaceutical composition can be administered to an individual suffering from neurodegenerative or neurodevelopmental disease. The neurodegenerative or neurodevelopmental disease can be selected from the group consisting of Alzheimer's disease, Huntington's disease, Parkinson's disease, Multiple Sclerosis, Autism spectrum disorders and brain neurodegenerative or neurodevelopmental syndromes originated therefrom, respectively, late or early stage human life, and brain Glutamatergic/GABA imbalance. The effective amount can be 50 to 300 mgs (po), administered daily to an individual suffering from the neurodegenerative or neurodevelopmental disease.

However, the above described preventive action of Fendiline must not be confused with its therapeutical neuroprotective action, against the symptoms and the evolution of the neurodegenerative diseases, as possible described in the prior art. Doses are different: 3 to 30 mgs for possible prior art and 50 to 300 mgs, for the preventive action of Fendiline described in this application. Moreover, these doses concerned, respectively, the partial sigma-1 agonism for possible prior art while sigma-1 antagonistic effects are concerned in the present application.

In conclusion, Fendiline, by the above pharmacological components, could develop a preventive efficient opposition against the initiation, step (1), and the amplification step (2), of the brain neuropathological processes (glutamatergic (NMDA) and interneuronal GABAergic and cholinergic aberrant hyperactivities), protecting against the step (3), (non-convulsive) seizures induced, cognitive deficits and neurodegenerative evolution of neurodevelopmental and neurodegenerative diseases.

While embodiments of the prototypical pharmacological profile of Fendiline have been described in detail, it should be apparent that modifications and variations thereto are possible, all of which fall within the true spirit and scope of the invention. And although against the new putative brain preventive properties against neurodegenerative and neurodevelopmental diseases have been described, it should be appreciated that the prototypical analgesic profile of fendiline herein described is also suitable for possible other treatments.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A compound comprising an effective amount of 3,3-diphenyl-N-(1-phenylethyl) propan-1-amine (Fendiline), its enantiomers and pharmaceutically acceptable salts or combinations thereof, said compound being selective sigma-1 receptor ligands and, therefore, prototypical allosteric antagonists of N-methyl-D-aspartate receptors [NMDA(−)], sodium channels [Nav(−)] and discreet desensitization agents of muscarinic receptors [M(−)], exercing also positive allosteric modulations on brain γ-aminobutyric acid (B) [GABA (B) (+)] and [GABA (A) (+)] receptors, said compound having a configuration with brain protective and preventive activities against neurodegenerative and neurodevelopmental diseases.

2. A pharmaceutical composition comprising an effective amount of a compound and at least one pharmaceutically acceptable excipient, said compound being selected from the group consisting of 3,3-diphenyl-N-(1-phenylethyl) propan-1-amine (Fendiline), enantiomers and pharmaceutically acceptable salts of Fendiline, and combination thereof, said compound being as prototypical sigma-1 ligand with [NMDA(−)], [Nav(−)], [M(−)], [GABA(B)(+)] and [GABA(A)(+)], said pharmaceutical composition having a configuration with brain protective and preventive activities against neurodegenerative and neurodevelopmental diseases.

3. The pharmaceutical composition of claim 2, wherein said effective amount is 50 to 300 mgs (po), daily.

4. A method of using a pharmaceutical composition in treating neurodegenerative or neurodevelopmental disease, said method comprising the step of administering to an individual suffering from neurodegenerative or neurodevelopmental disease an effective amount of a pharmaceutical composition comprising an effective amount of a compound and at least one pharmaceutically acceptable excipient, said compound being selected from the group consisting of 3,3-diphenyl-N-(1-phenylethyl) propan-1-amine (Fendiline), enantiomers and pharmaceutically acceptable salts of Fendiline, and combination thereof, said compound being as prototypical sigma-1 ligand with [NMDA(−)], [Nav(−)], [M(−)], [GABA(B)(+)] and [GABA(A)(+)].

5. The method of claim 4, wherein said neurodegenerative or neurodevelopmental disease being selected from the group consisting of Alzheimer's disease, Huntington's disease, Parkinson's disease, Multiple Sclerosis, Autism spectrum disorders and brain neurodegenerative or neurodevelopmental syndromes originated therefrom, respectively, late or early stage human life, and brain Glutamatergic/GABA imbalance.

6. The method of claim 4, wherein said effective amount of said compound is 50 to 300 mgs (po), daily.
